(19) United States
(12) Patent Application Publication
(10) Pub. No.: US 2011/0184439 A1
(43) Pub. Date: Jul. 28, 2011

(54) Biological Matrix for Cardiac Repair

(75) Inventors: William D. Anderson, Pittsburgh, PA (US); Stephen F. Badylak, Pittsburgh, PA (US); Thomas W. Gilbert, Pittsburgh, PA (US); John M. Wainwright, Churchill, PA (US)

(73) Assignee: University of Pittsburgh-Of the Commonwealth System of Higher Education, Pittsburgh, PA (US)

(21) Appl. No.: 12/991,809

(22) PCT Filed: May 8, 2009
(86) PCT No.: PCT/US09/43264

§ 371 Date: Apr. 8, 2011

(51) Int. Cl. A61B 17/08

(52) U.S. Cl. 606/151

(57) ABSTRACT

Provided herein is a device to occlude a hole in a wall of an organ or tissue. In another embodiment, a device is provided which comprises an extracellular matrix-derived material and an adhesive to occlude a hole in a wall of an organ or tissue. Provided are devices prepared from extracellular matrix-derived cell-growth scaffolding to repair defects in walls of organs or tissues. Also provided are methods for preparing the device as well as for using the device.

### CROSS REFERENCE TO RELATED APPLICATIONS

This application is a National Stage of International Application No. PCT/US2009/043264, filed May 8, 2009, which in turn claims the benefit of U.S. Provisional Application No. 61/051,734, filed May 9, 2008, each of which is incorporated herein by reference in its entirety.

### STATEMENT REGARDING FEDERAL FUNDING

This invention was made with government support under Grant No. R43 HL083627-01, awarded by the National Institutes of Health. The government has certain rights in this invention.

Defects within the heart include holes between the upper chambers of the heart (atrial septal defects or ASD) and between the lower chambers of the heart (ventrical septal defects or VSD). Approximately 25% of the general population has an ASD called patent foramen ovale (PFO). Diagnosis with PFO indicates an improperly closed foramen ovale, a passageway between the left and right atria in the fetal heart, which leaves a small hole in the septum between the atria. PFO has been correlated with strokes, atrial septal aneurysm and migraine headaches. The prevalence of migraines in the United States is approximately 10% (or 28 million of the general population) and about 3 million of those patients are believed to have PFO.

Cardiac septal defects often can be treated with implants that are delivered through minimally invasive procedures, such as delivery through catheters or other endoscopic approaches. Many of these implants are flexible and collapsible, so that the collapsed device can be attached to the end of the catheter or pushed through the lumen of the catheter by a guide wire. To treat cardiac septal defects, the catheter is typically inserted through a large vein and into the right ventricle of the heart. In the case of patent foramen ovale, the catheter is guided towards the defect in the atrial septum and the device is deployed or expanded to cover up the defect.

Implants currently used to correct ASD are composed of biocompatible, yet non-degradable materials, such as metals and polytetrafluoroethylene (PTFE). Though non-degradable implants are used to repair cardiac defects, those implants can also interfere with future medical procedures that require access to the left atrium by punching through the septum of the heart. Frequently, implants need to be replaced due to dislodgement from the defect or erosion of the device itself. A patient also may require a larger implant if the defect enlarges over time. The dimensions of the device must be pre-determined by assessing the size of the defect and of the vasculature of the patient. For example, percutaneous procedures for children require smaller catheters and smaller devices than procedures for adults. If the size of the defect is mis-judged, or the patient too small at the time of implantation, the patient may grow out of the device. Thus, there is a need for a device capable of being easily removed.

The CardioSEAL® and STARflex® Occluders (both commercially available from NMT Medical) have a metal alloy frame with polyester fabric attached. CardioSEAL® has an MP35n frame (nickel-cobalt-chromium-molybdenum alloy). The STARflex® product has a self-centering system composed of coil microsprings. BioSTAR® (commercially available from NMT Medical) has the same framework as STARflex® but has a biodegradable acellular collagen matrix rather than the polyester fabric. About 90-95% of the BioSTAR® implant is absorbed and replaced with native and scar tissue. However, these devices all suffer from the critical defect of not being able to be easily removed. Specifically, such devices once implanted cannot change shape allowing for easy removal. Thus, there is a critical need for removable implant devices

### SUMMARY

Provided herein is a device for occluding a defect in a tissue such as a septal wall in a patient. Such a device comprises an occluding member comprising a collapsible frame, the frame comprising a distal sealing portion, a proximal sealing portion, and a connector between the sealing portions comprising a plurality of shape memory fibers extending from the proximal portion to the distal portion and formed into a preset shape of a twisted bundle, and which can be untwisted by rotating one or both of the proximal and distal portions relative to each other. Further, the device may further comprise a fastener that facilitates manipulation and retrieval of the device. The fastener can be, without limitation, a threaded bore or a bolt from a bolt-and-nut type clasp or an eye or hook of a hook-and-eye-type clasp.

The device may have any useful shape or configuration. For example and without limitation, the proximal and distal sealing portions may be cambered. The proximal and distal sealing portions may comprise eyelets and graft materials including for example, ECM-derived material may be attached to the eyelets on the proximal portion of the frame. When present, the eyelets can be configured to allow for manipulation and retrieval of the device and may further comprise sutures. The connector between the proximal and distal portions of the frame may have any useful configuration, and may comprise one or more shape memory fibers. For example and without limitation, the connector may consist of a single spring-shaped memory fiber. In another non-limiting example, the connector comprises a plurality of shape memory fibers extending from the proximal to the distal portion and formed into a preset shape of a twisted bundle, and which can be untwisted by rotating one or both of the proximal and distal portions relative to each other. The occluding member of the device may further comprise a medically-acceptable adhesive, such as, without limitation fibrin and/or a cyanoacrylate.

According to one embodiment of the technology described herein, a device is provided for occluding a defect in a wall in a patient comprising an occluding member having any medically compatible graft material, including for example, an extracellular matrix (ECM) derived material. The defect can be, without limitation, an atrial septal defect, a patent foramen ovale, a cardiac rupture, a tracheal-esophageal anastomosis, a gastric anastomosis, or a gastric ulcer. In one non-limiting embodiment, the ECM-derived material is laminar and comprises one or more layers of ECM tissue and can be isolated from any useful tissue source, for example and without limitation, from urinary bladder tissue, intestinal submucosa, small intestinal submucosa, dermis of skin and/or heart. In one non-limiting example, the ECM tissue comprises epithelial basement membrane and subjacent tunica propria, and in one embodiment, substantially comprises epithelial basement membrane and subjacent tunica propria. The ECM tissue may be oriented so that when the device is installed in the wall, the epithelial basement membrane (luminal surface) of the ECM tissue is exposed. In another embodiment, the ECM tissue comprises epithelial basement membrane, subjacent tunica propria, and tunica submucosa. The ECM tissue may further comprise one or both of tunica muscularis and tunica submucosa.

Multiple layers of ECM tissue may be used in the device. For example and without limitation, from 2 to 20 layers of extracellular matrix tissue may be used. The ECM tissue of the device may be seeded with cells, such as, without limitation: human cells, autologous or allogeneic cells, which may be progenitor cells (precursor cells), such as stem cells. The cells may be endothelial cells. In certain non-limiting embodiments, the device may comprise a hydrogel prepared from comminuted ECM tissue. In certain non-limiting embodiments, the device may comprise radiopaque material.

A method of repairing a defect in a wall in a patient also is provided. The method comprises delivering a device comprising an occluding member, in any of its possible variations described above and throughout this document, to the site of a defect in a patient and occluding the defect with the device. In one non-limiting embodiment, the defect is a cardiac septal defect, such as an ASD. As non-limiting examples, the occluding member may comprise a collapsible frame and the device is delivered to the site of the defect in a collapsed state. In another non-limiting embodiment, the occluding member further comprises an adhesive and the device is placed about the defect to occlude the defect. The device may be delivered through a catheter or trocar.

According to another embodiment, a method of making any device in any embodiment described above or throughout this document, comprising attaching an ECM-derived material to a frame of a device for occluding a defect in a wall. In one non-limiting example, the prepared ECM-derived material is laminar. In another, the method comprises attaching the prepared ECM-derived material to a collapsible frame. In yet another, the method comprises applying an adhesive to the prepared ECM-derived material.

A kit for use in repair of a defect in a wall in a patient comprising a device in any embodiment described above or throughout this document, comprising an occluding member and frame in a container (in suitable packaging, acceptable for transport and storage of implantable medical devices). The ECM-derived material may be dehydrated. The device may be connected to a guide wire or a guiding catheter. The kit may further comprise a delivery catheter, cannula and/or trocar. The kit may further comprise a funnel to draw the device into the delivery mechanism. The kit may further comprise an ECM-derived hydrogel in a commercially and medically acceptable container.

### BRIEF DESCRIPTION OF THE DRAWINGS

FIG. 1 schematically shows transcatheter delivery of one non-limiting embodiment of an occluding device through the inferior vena cava and into a human heart with an atrial septal defect.

FIGS. 2A-2D schematically show deployment of one non-limiting embodiment of an occluding device to occlude a defect in a wall.

FIG. 3 is a schematic of a cross-sectional view of the wall of the urinary bladder (not drawn to scale). The following structures are shown: epithelial cell layer (A), basement membrane (B), tunica propria (C), muscularis mucosa (D), tunica submucosa (E), tunica muscularis externa (F), tunica serosa (G), tunica mucosa (H), and the lumen of the bladder (L).

FIGS. 4A-4D show the structure of one non-limiting embodiment of an ECM-derived sheet as used in an embodiment of a device described herein. FIG. 4A is a photograph of a porcine urinary bladder matrix—derived material in a lyophilized sheet form. FIG. 4B is a schematic diagram of ECM-derived material in laminar form, where multiple sheets are laminated together. FIG. 4C is an exploded schematic view of one embodiment of a device described herein. FIG. 4D is a perspective schematic view of one embodiment of a device described herein.

FIGS. 5A-F schematically show a connector comprising a plurality of shape memory fibers as in one non-limiting embodiment of a device described herein. FIG. 5A is a schematic diagram of the connector when stress is applied to pull apart the proximal and distal portions of the device. FIG. 5B is a schematic diagram of the connector when the stress is not applied to the proximal and distal portions of the device and the fibers assume its preset shape of a twisted bundle. FIG. 5C is a close-up view of the connector shown in FIG. 5A. FIG. 5D is a close-up view of the connector shown in FIG. 5B in its preset shape. FIGS. 5E and 5F is a schematic diagram showing the connector accommodating defects of different tunnel lengths.

FIGS. 6A-D schematically show a frame with straight struts as used in one non-limiting embodiment of a device described herein. FIG. 6A shows the top and side views of a frame with parallel struts. FIG. 6B shows a perspective view of the frame with parallel struts. FIG. 6C shows the top and side views of a frame with staggered struts. FIG. 6D shows a perspective view of the frame with staggered struts.

FIGS. 7A-D schematically show a frame with curved struts as used in one non-limiting embodiment of a device described herein. FIG. 7A shows the top and side views of a frame with parallel struts. FIG. 7B shows a perspective view of the frame with parallel struts. FIG. 7C shows the top and side views of a frame with staggered struts. FIG. 7D shows a perspective view of the frame with staggered struts.

FIGS. 8A-C schematically show a frame with a helical periphery as used in one non-limiting embodiment of a device described herein. FIG. 8A shows a collapsed device comprising a helical frame and an ECM-derived material. FIG. 8B shows the device deployed from the catheter. FIG. 8C shows the device as installed within a septal defect.

FIGS. 9A-C schematically show a frame with double occlusion discs according to one non-limiting embodiment of a device described herein. FIG. 9A shows a collapsed device comprising double discs and an ECM-derived material. FIG. 9B shows the device deployed from the catheter. FIG. 9C shows the device as installed within a septal defect.

FIGS. 10A-B schematically show a patch to repair a cardiac rupture as used in an embodiment of a device described herein. FIG. 10A shows the heart with a cardiac rupture, namely, a free wall defect. FIG. 10B shows the patch being used to occlude the free wall defect.

FIGS. 11A-B schematically show a non-limiting example of a set of forming tools to preset the structure of a frame. FIG. 11A shows a set of two plates used to form the struts and eyelets of the frame. FIG. 11B shows a set of two plates used to introduce twists into the connector.

FIGS. 12A-12B schematically show a non-limiting example of a set of forming tools to compress the connector portion of the frame. FIG. 12A shows a set of three plates, where the top and bottom plates maintain the shape of the eyelets and struts of the frame and the middle plate contains the connector. FIG. 12B shows the set of three plates being held together to compress the connector.

FIG. 13: Shown is a NiTi frame in expanded and compressed states.

FIG. 14: Shown is a NiTi frame with the grafting material (UBM-ECM) attached.

FIG. 15: Shown is a trans-esophageal echocardiogram of the atrial septal defect (ASD) area patched with the occluding device of Example 3, one week post surgery 15A and controls 15B (color doppler no shunt) and 15C (saline bubbles with no shunt).

FIG. 16: Shown is an epicardial echocardiogram of the ASD area patched with the UBM device of Example 3, three months post surgery (16A) and control (16B).

### DETAILED DESCRIPTION

The devices and methods provided herein are used for occluding holes and defects found within the tissues or organs in a patient. In certain embodiments, the defect is a cardiac defect affecting the atria, ventricles or septum. In one non-limiting example, the defect is an atrial septal defect or a patent foramen ovale. In another embodiment, the defect is a cardiac rupture. In yet another embodiment, the defect is any defect accessible with an endovascular procedure. In another embodiment, the defect is any defect accessible with a transcatheter or endoscopic procedure, such as, without limitation, a tracheal-esophageal anastomosis, gastric anastomosis, or gastric ulcer.

The use of numerical values in the various ranges specified in this application, unless expressly indicated otherwise, are stated as approximations as though the minimum and maximum values within the stated ranges are both preceded by the word “about.” In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. All references are fully incorporated by such reference herein, solely to the extent of their technical disclosure and only such that it is consistent with this disclosure.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “subject” refers to members of the animal kingdom including but not limited to human beings that are treated using the methods and compositions described herein.

“Treatment” of a medical condition associated with a heart defect and/or injury means administration to a subject by any suitable route a device that can repair the defect/injury with the object of ameliorating (e.g., attenuating, alleviating, reducing and/or normalizing) any symptom and/or indicia associated with the medical condition, including, without limitation, any testable parameter, whether or not subjective. Likewise “treating” such a medical condition may result in amelioration of any symptom and/or indicia associated with the medical condition in a subject.

As used herein, the term “defect(s)” or “hole(s)” refers to any type of damage found in the tissues or organs in a patient. Damages include those resulting from any number of circumstances, such as, without limitation, injuries, ischemia, infarct, congenital defects, disease, infections and other acquired illnesses. For example and without limitation, cardiac defects include atrial septal defects (ASD), patent foramen ovale (PFO), ventrical septal defect, free wall rupture and any holes found within the cardiac tissue.

For closure of cardiac defects, the device may be delivered to the site of the defect using any one of many medically accepted procedures and preferably a minimally-invasive procedure, such as a transcatheter or endoscopic procedure. When compared to open heart surgery, transcatheter or endoscopic procedures are less invasive and have comparable clinical outcomes. In one embodiment, a trocar is inserted beneath the xyphoid process to access the left ventricle. A trocar is a hollow, cylindrical surgical instrument with a sharp point. Upon inserting the trocar into the patient, cannulas and other medical equipment or devices can be passed through the trocar to access blood vessels or body cavities.

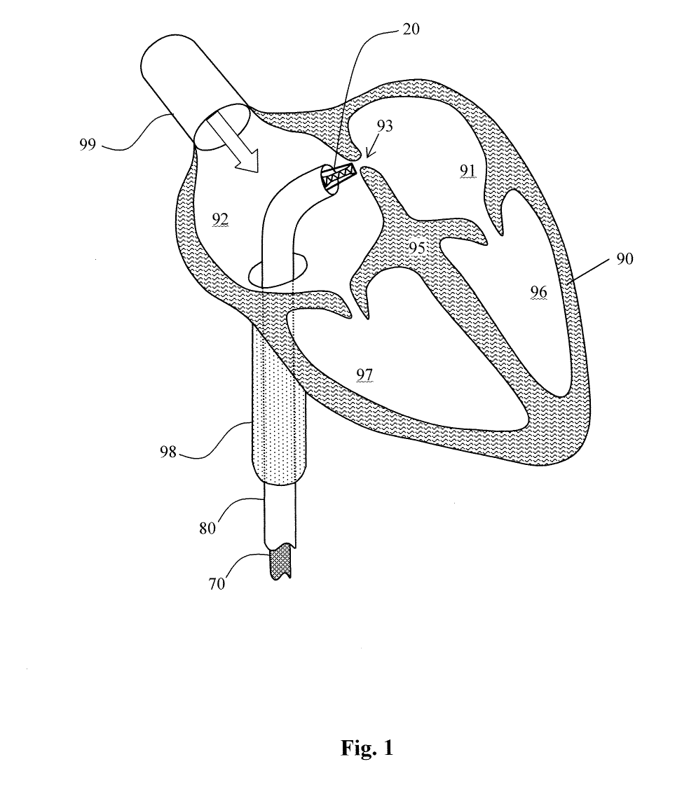

FIG. 1 depicts one non-limiting embodiment, in which a device 20 is delivered to the heart using a transcatheter procedure. A catheter 80 is inserted into, for example, a femoral vein and is guided through the inferior vena cava 98 to the heart 90. A catheter is a hollow tube that is pushed into a body cavity, duct or vessel and used to drain fluids, inject fluids or drugs, and to deliver medical devices. Typically, a catheter is a long, hollow tube with a lumen of a small inner diameter and a luer lock on the proximal (closest to the operator of the catheter) end. A proximal segment of the catheter can be more rigid to allow pushing of the catheter, while the distal (farthest from the catheter operator, first inserted into the patient) end of the catheter can be more flexible to minimize vessel trauma. Device 20 includes a guide wire 70 and is delivered through the catheter 80. In another embodiment, the device 20 can be attached to another catheter that has a smaller diameter than the delivery catheter 80.

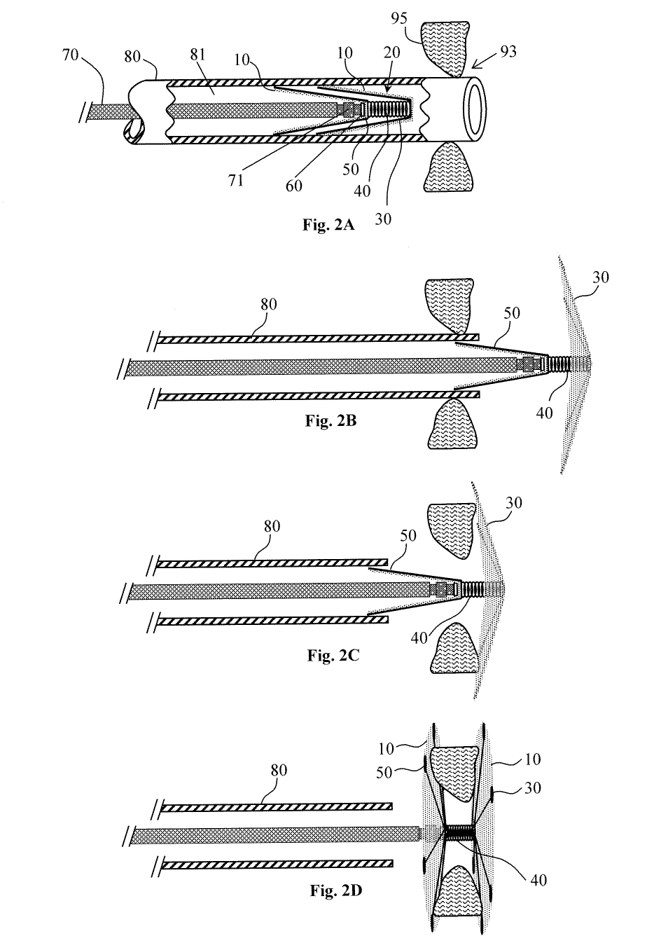

The device 20, shown in FIG. 1, is used to treat patent foramen ovale 93, where the catheter 80 is further guided into the right atrium 92 and into the defect 93 in the septum 95 of the heart 90 (FIG. 1). In FIG. 1, the superior vena cava 99, inferior vena cava 98, left atrium 91, right atrium 92, left ventricle 96 and right ventricle 97 are shown for reference. In one non-limiting embodiment shown in FIGS. 2A-2D, the device 20 comprises grafting material 10which closes the hole (e.g., ECM-derived material), distal sealing portion 30, connector 40, proximal sealing portion 50, and fastener 60. As illustrated in FIGS. 2A-D, the folded or non-deployed device 20 is pushed through the lumen 81 of the catheter 80 with guide wire 70. The device is connected to the locking mechanism 71 on the guide wire 70 by the fastener 60. Then, the distal portion 30 of the device 20 is deployed in the left atrium (FIG. 2B). By maintaining the position of the guide wire 70 and then slowly pulling the catheter 80 away from the device, only the distal portion 30 of the device can be exposed and therefore deployed. The device is repositioned within the defect, wherein the connector 40 spans the tunnel length of the defect (FIG. 2C). The catheter 80 is then further pulled to expose and to deploy the proximal portion 50 in the right atrium (FIG. 2D). The device is installed by releasing it from the guide wire 70 (not shown). In one embodiment, the locking mechanism 71 of the guide wire 70 is released from the fastener 60 of the device, wherein the fastener 60 is a threaded bore. In another embodiment, the fastener 60 can be a hook. The fastener can be engaged when the device needs to be repositioned or retrieved later on.

For adequate closure of the cardiac defect, for example and without limitation as shown in FIGS. 1 and 2A-2D, the sealing portions should be large enough to cover the size of the defect and the connector of the device should be long enough to traverse the tunnel length of the defect. For ease of delivery through a catheter or other device having a lumen, and to accommodate smaller vasculature, the device typically is capable of being folded or otherwise compressed before and during deployment. In one non-limiting embodiment, the non-deployed device has a diameter of less than 4 mm and a diameter of from about 1 cm to about 6 cm when deployed. In another embodiment, the device comprising the matrix is stored in an expanded condition and wetted to slide, folded or otherwise compressed, through a catheter. In yet another embodiment, the device comprising the ECM-derived matrix and a frame is stored in a non-deployed condition.

During deployment of the device in a patient, the position of the device in a patient may be confirmed through medical imaging techniques, such as x-ray and fluoroscopic visualization. Radiopaque markers can be incorporated into the device to facilitate the imaging process. Relevant radiopaque markers include, without limitation, gold, platinum, zirconium oxide and barium sulfate markers. In one embodiment, all or part(s) of the frame and/or bioscaffold are coated with a radiopaque material.

In one embodiment, the connector is a variable-length connector which comprises screw (including bolts, namely a cylindrical structure having a threaded portion or shaft comprising helical or spiral threads in any useful variation) that can be turned via the catheter. The screw is attached to the proximal and distal sealing portions in a manner that by turning the screw, the distance between the proximal and distal sealing portions can be increased or decreased. In one embodiment, the head of the screw is caged in a portion of the proximal portion in a manner which permits the screw to be turned and holds the head in place relative to the proximal portion, and a threaded portion of the screw passes through a nut or other tapped structure for engaging the threaded portion in any useful manner which travels along the threaded portion of the screw when the screw is turned and thereby increases or decreases the distance between the distal sealing portion and the proximal sealing portion. In a typical installation, prior to insertion in a hole, the screw is turned so that the connector is in an elongated configuration and the proximal and distal sealing portions are at an extended distance from each-other. The device is deployed in a hole, with the proximal and distal sealing portions deployed on opposite sides of the hole (see, e.g., FIGS. 2A-2D) and the screw is turned to shorten the connector and decrease the distance between the proximal and distal sealing portions, thereby compressing the proximal and distal sealing portions about the hole.

The frame can be formed from a variety of materials or combinations thereof. In one embodiment, a portion or portions of the frame comprise ECM-derived material. Layers of sheets of ECM-derived material can be laminated together using various methods known in the art, including without limitation, treatment by vacuum-pressing, chemical bonding through cross-linking with carbodiimide or isothiocyanate or photooxidation methods, non-chemical bonding by dehydrothermal methods. The laminar material can be further cut and shaped into any portion or portions of the frame, including without limitation, struts, eyelets, or connectors.

The frame or portions thereof may comprise a biocompatible alloy or polymer. The frame of portions thereof may comprise a biocompatible shape memory alloy or polymer. Examples of shape memory alloys include, without limitation, nitinol and cobalt-alloys. Examples of biocompatible shape memory polymers include, without limitation, homopolymers and copolymers comprising PLLA (poly-L-lactic acid), PGA (polyglycolic acid), polycarbonates, and methacrylates.

As used herein, the terms “shape-retaining” and “shape memory” refers to the quality of a material to return to a preset, “resting” or low energy shape upon a stimulus, such as a change in temperature, wavelength of light or mechanical stress. For example and without limitation, nitinol is a shape memory metal alloy, where heating beyond the transition temperature sets the shape of the nitinol. While applying mechanical stress will deform nitinol from its preset state, removing the stress will return nitinol to its preset shape. Due to this characteristic, nitinol are said to be elastic or superelastic or pseudoelastic. In another example, without limitation, a co-polymer of oligo(E-caprolactone) dimethacrylate and n-butyl acrylate is a shape memory polymer, where heating the polymer past a transition temperature returns it to a preset shape.

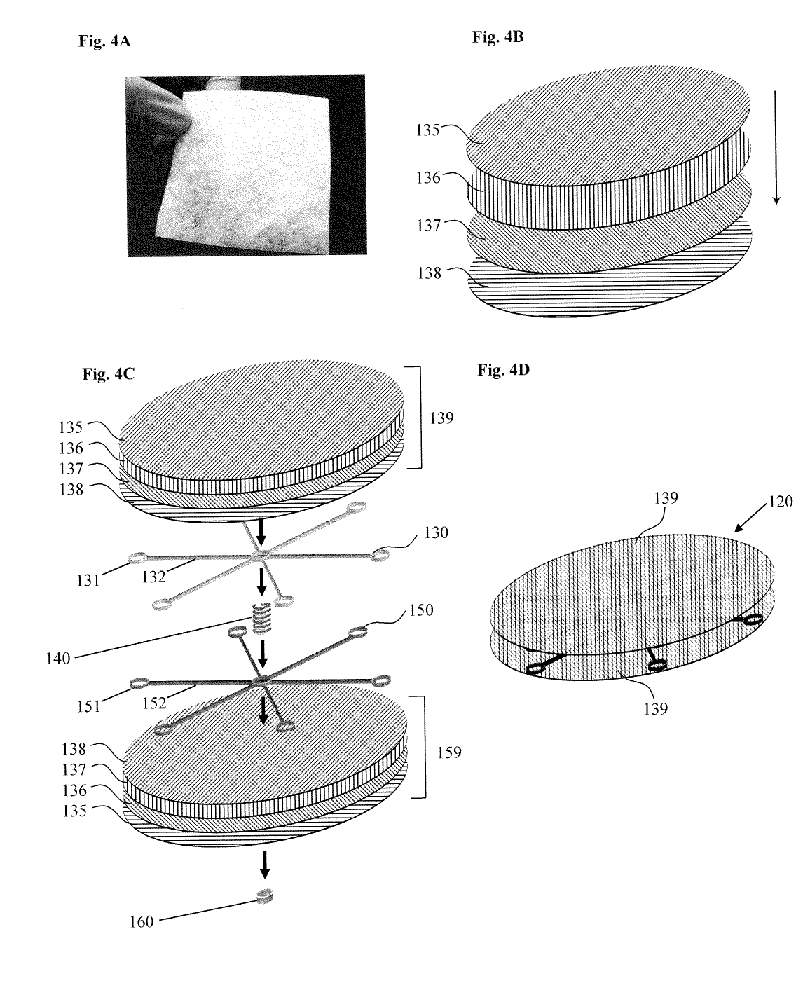

As described above, according to certain embodiments of the device described herein, the frame comprises a fastener that facilitates placement of the device and retrieval, if necessary. FIG. 4C depicts one embodiment of fastener 160. In one embodiment, the fastener comprises a threaded bore or nut and the locking mechanism comprises a bolt configured to engage the threaded bore and the fastener and the locking mechanism are parts of a bolt-and-nut clasp system. In another embodiment, the fastener comprises an eye attached to sutures run through the eyelets and the locking mechanism comprises a hook, wherein the fastener and the locking mechanism are parts of a hook-and-eye clasp system.

In use, the fastener allows for the frame to be retrievable or repositionable. The locking mechanism of the guide wire can be pushed through a catheter to an implanted device. Then, the locking mechanism attached to the guide wire is inserted into the fastener of the device. If the fastener is a threaded bore, then the locking mechanism comprises a bolt that can be screwed into the bore. If the fastener is an eye, then the locking mechanism comprises a hook that can latch into the eye.

In the device, the connector between distal and proximal frame portions of the device can comprise a variety of configurations. In one embodiment, the connector is variable-length and comprises a spring, helix or other structure consisting of one or more fibers of an alloy or polymer or a shape-retaining material. When the device is collapsed, for instance in a catheter, the spring will deform and become elongated. Deploying the device will remove the mechanical stress on the spring and allow the spring to return to its preset configuration causing compression between sealing members attached to the connector. For example and without limitation, the spring's preset configuration can be a spiral with a certain pitch and diameter.

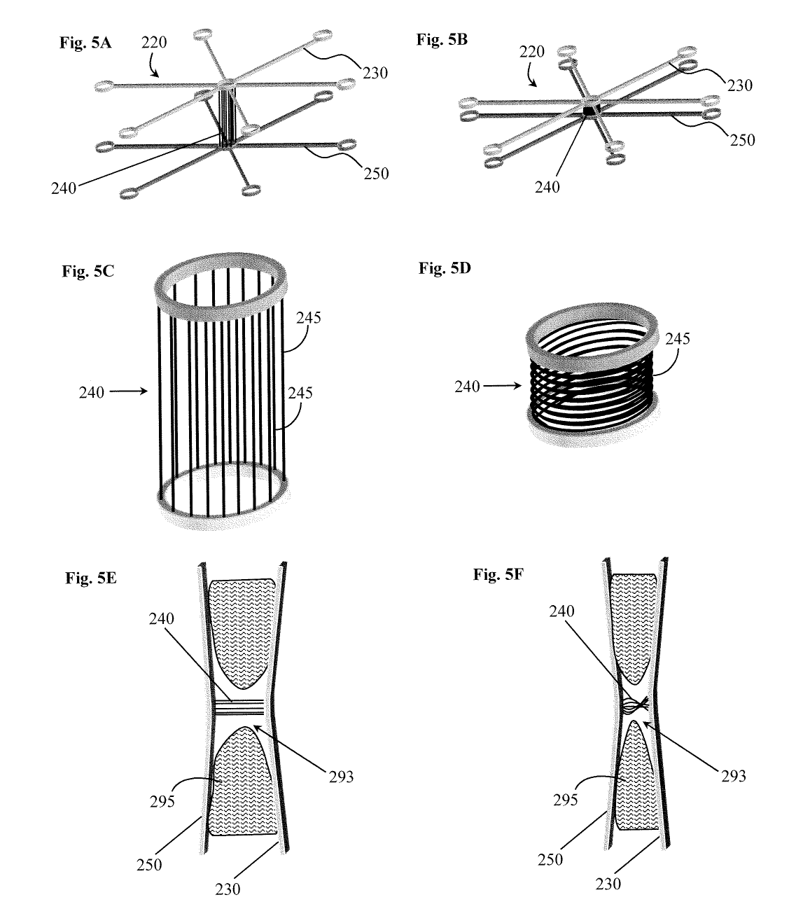

In another embodiment, and in reference to FIGS. 5A-5F, the connector 240 of the device 220 comprises a plurality of shape memory fibers 245 extending between the distal 230 and proximal 250 portions of the frame, and formed into a preset shape of a twisted bundle, which can be untwisted by rotating one or both of the proximal 250 and distal 230 portions relative to each other. By unwinding the twist in the connector 240, the distance between the proximal 250 and distal 230 portions can be increased. Because connector 240 is made from a shape memory material, such as nitinol, when the fibers 245 are distorted from their preset shape by untwisting, proximal 250 and distal 230 portions will exert an inward pressure (towards each-other), pressing the proximal 250 and distal 230 portions against walls surrounding a defect into which the device is implanted. Before assembling the device 220, the plurality of fibers 245 is heat treated in methods known in the art to the preset configuration shown in FIGS. 5B and 5D. For example and without limitation, each fiber 245 is treated to have a preset twisted configuration, where the twists of a plurality of fibers 245 form one bundle.

In use, the connector 240 comprising a plurality of shape memory fibers 245 can assume different configurations. Varying the distance between the distal 230 and proximal 250 frame portions of the device varies the amount of mechanical stress applied to the fibers and the amount of mechanical stress on the fibers determines the configuration of the fibers. In reference to FIGS. 5A and 5C, the distal 230 and proximal 250 frame portions are pulled apart to exert the maximum amount of stress upon the fibers 245. When the maximum amount of stress is applied to the twisted plurality (or bundle) of fibers, the fibers become elongated and substantially straight (linear), and are approximately parallel to one another. The maximum amount of stress is defined as the amount of stress than can be applied until the fiber breaks or loses some other mechanical or physical property. In reference to FIGS. 5B and 5D, when the distal 230 and proximal 250 frame portions are allowed to relax to remove mechanical stress, the fibers 245 assume the preset twisted configuration. As the shape memory fibers can assume various configurations based on the amount of stress applied, these fibers are considered elastic. In reference to FIGS. 5E and 5F, the elasticity of the fibers allows the connector to accommodate various tunnel lengths of the defect. Increasing the tunnel length of a defect increases the distance between the distal 230 and proximal 250 frame portions, which results in higher mechanical stress. As a result, a connector that spans a larger defect is less compressed than a connector spanning a smaller defect.

In one embodiment, the plurality of fibers comprises a biocompatible shape memory alloy, including without limitation, nitinol and cobalt-alloys. In another embodiment, the plurality of fibers comprises a biocompatible polymer, including without limitation, homopolymers and copolymers comprising polylactides such as PLLA (poly-L-lactic acid;), PGA (polyglycolic acid), polycarbonates, and methacrylates. In yet another embodiment, absorbable metal is used in the fibers including, for example, magnesium (Mg) alloys (e.g., Mg with yttrium and rare earth additives).

Therefore, according to certain embodiments of the devices described herein, a device is provided having proximal and distal sealing portions connected to each-other via a variable-length connector. The connector between the proximal and distal portions of the frame may have any useful configuration, and may comprise one or more shape memory fibers. According to certain embodiments, the connector has an extended and resting state (a lower-energy preset state or shape), wherein in the extended state, the connector is longer than in the resting state. As such, the distance between the proximal and distal portions of the frame is greater when the connector is in its extended state, and less when the connector is in its resting or preform state. Further, when the connector is in its extended state, the connector pulls the proximal and distal portions of the frame towards each-other, which, when in use to seal a hole, causes compression on both sides of the tissue surrounding the hole. In certain embodiments, the connector comprises a shaped memory material such as one or more fibers. The connector can have any preset shape/configuration, including spiral, helix, spring, etc., as well as any extended configuration. In one non-limiting example, the connector has a spiral or helical (e.g., spring) preset shape. In another non-limiting example, the connector comprises a plurality of shape memory fibers extending from the proximal to the distal portion and formed into a preset shape of a twisted bundle or spring which can be untwisted by rotating one or both of the proximal and distal portions relative to each other. The distance between the proximal and distal portions can be extended manually during insertion of the device in the hole, for instance by twisting the proximal and distal sealing portions relative to each other prior to or during insertion into a catheter for deployment, or by twisting during insertion at the deployment location. Twisting of the proximal and distal sealing portions relative to each-other can be accomplished by a variety of means, such as by use of a wire deployed through the deployment catheter.

Thus, more generally, according to certain non-limiting embodiments of the device described herein, a device for sealing holes in a tissue, system, organ, etc. (structure) in a patient is provided, the device comprising proximal and distal sealing portions and a variable-length connector. Such a device can be used to repair any tissue described herein or known in the art to be amenable to repair using such methods and devices. The length of the variable-length connector can be controlled by any useful method, including use of screwing mechanisms, shape-retaining materials, springs, or even by use of a loop or loops of a fiber which can be pulled and tied off or otherwise locked into a compressed configuration. The device is deployed about a hole in a structure in a patient with the connector in an extended configuration, and, once the proximal and distal sealing portions are deployed about the hole, the connector is shortened, thereby compressing the proximal and distal sealing portions about the hole.

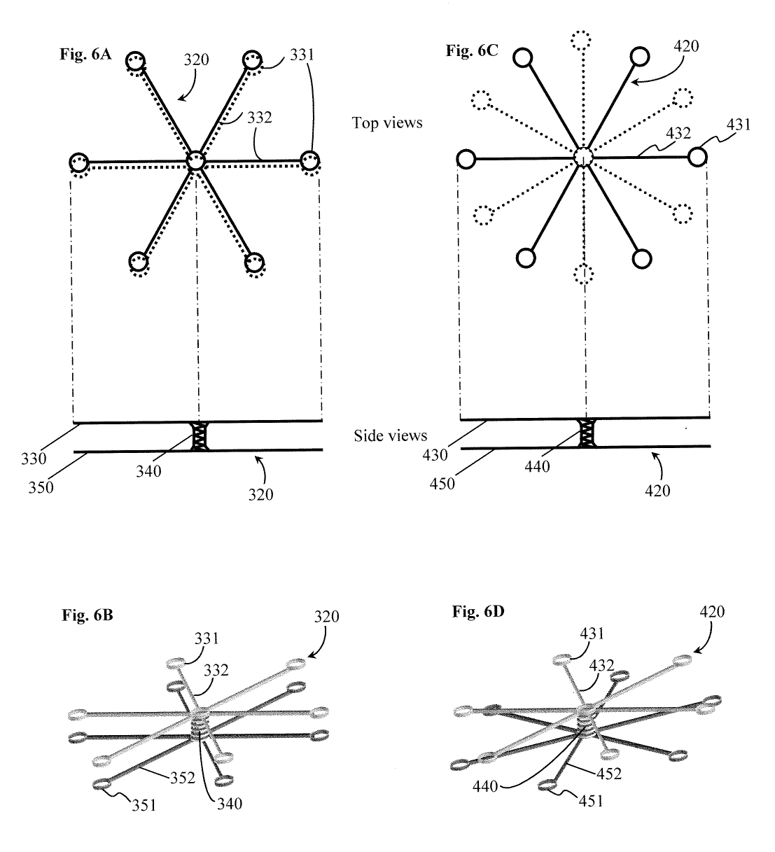

The configuration of the proximal and distal sealing portions of the frame can be optimized to provide suitable coverage of the defect. In reference to FIGS. 6A and 6B, according to one non-limiting embodiment of the device described herein, the device 320 comprises a distal sealing portion 330 with eyelets 331 and straight struts 332 and a proximal sealing portion 350 with eyelets 351 and straight struts 352, wherein the distal struts 332 are substantially parallel to (aligned with) the proximal struts 352 (as shown in phantom in FIG. 6A). In another non-limiting embodiment shown in FIGS. 6C and 6D, the device 420 comprises distal struts 432 that are staggered with respect to the proximal struts 452 (as shown in phantom in FIG. 6C). The number of struts per frame can vary. For example and without limitation, a frame can have 4, 5, 6, 7, 8, 9, 10 or more for each of the sealing portions. The struts can be curved (not shown).

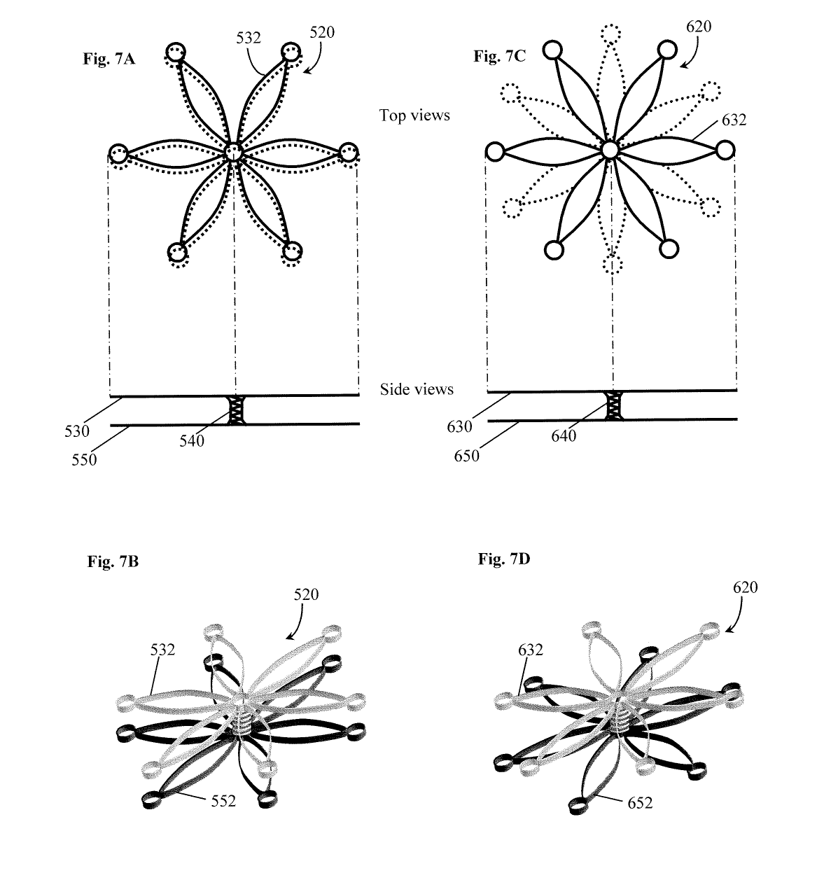

The geometry of the struts can be optimized to provide superior structural integrity to the frame or support for the grafting material being attached to the frame. In one embodiment shown in reference to FIGS. 7A and 7B, the device 520 comprises a distal sealing portion 530 with curved struts 532 and a proximal sealing portion 550 with curved struts 552, wherein the distal struts 532 are parallel to (aligned with) the proximal struts 552 (as shown in phantom in FIG. 7A). Each curved strut comprises two arcs that form an oval with the center of the device and the eyelet at the ends of the major axis of the oval. In another embodiment shown in FIGS. 7C and 7D, the device 620 comprises distal struts 632 are staggered to the proximal struts 652. In yet another embodiment, U-shaped wires can be added to each side of the frame portions to provide additional support and to aid in wall apposition.

In one non-limiting embodiment, the struts of the frame of the device are made from nitinol wire, ranging in diameter from 0.005″ to 0.015″; typically 0.007-0.010″. The wire may be formed into the shape shown in, for example and without limitation, FIG. 5B, through multiple heat treatment steps. In one non-limiting embodiment, the wire is SE-508 with an Austenite Final (AF) temperature below 30° C. The wire is made corrosion resistant through electropolish, forming a titanium oxide coating on the surface. The purpose of these struts is to facilitate placement and fixation of the device during initial deployment and maintain a flattened or near flattened form of the device such that it serves as an effective barrier in the atrial septum.

The proximal and distal sealing portions of the frame should be sufficiently parallel with the walls of the septum. In one embodiment, the proximal and distal portions of the device are cambered outward to maximize coverage of the defect and minimize disturbance of blood flow within the heart. As used herein, the term “cambered” refers to the curved geometry of the proximal and distal portions of the device. More specifically, the term “cambered outward” refers to the proximal and distal portions curved in an expanded configuration such that the distance between the centers of the two portions is less than the distance between some or all peripheral edges of the two portions.

Commercially-available frames can also be used to make the device wherein the graft material can be in sheet or laminar or hydrogel form. Commercially available medical devices include, but are not limited to: CardioSEAL®, STARflex®, and BioSTAR® Occluders (NMT Medical); GORE HELEX Septal Occluder (W. L. Gore and Associates, Inc.); AMPLATZER® Septal Occluder, PFO Occluder, and Duct Occluder (AGA Medical Corp.).

Devices and methods are described herein for the preparation and use of a grafting material such as, polyester, metal, plastic, biodegradable polymers, ECM-derived (extracellular matrix-derived) cell-growth scaffolding, et alia, within devices to repair defects in walls of organs or tissues, such as without limitation, the heart. In certain embodiments, the ECM-derived scaffolding may be obtained from any suitable tissue. As used herein, the terms “extracellular matrix” and “ECM” refer to a complex mixture of structural and functional biomolecules including, but not limited to, structural proteins, specialized proteins, proteoglycans, glycosaminoglycans, and growth factors that surround and support cells within mammalian tissues. “ECM derived” is intended to mean that the graft material is made from in part or in whole from ECM. The ECM-derived matrix stimulates growth of the patient's tissues within the defect while it degrades. ECM-derived bioscaffolds are immediately recognized by host cells within the blood and surrounding tissues. These cells participate in a remodeling process that includes degradation of the ECM-derived scaffold and deposition of new matrix by the host cells that infiltrate the scaffold. The new matrix becomes the repair tissue over a period of time ranging from weeks to months, typically within 60-90 days. The end result of the process is host tissue filling a defect with functional tissue that otherwise would not be filled.

The tissue remodeling process stimulated by the ECM-derived matrix promotes the growth of tissue that has the function and morphology of native tissues at that site. Therefore, the matrix minimizes or eliminates the formation of non-functional scar tissue. During the tissue remodeling process, an ECM-derived matrix degrades and there is minimal foreign material remaining within the patient. In addition, dislodgment of the device will not be a problem because native tissue fills in the defect. Future medical procedures in a patient receiving the device that require access through the septum will not be impeded in many instances.

Any type of biocompatible polymer can be used to make the device described herein. Thus, it is contemplated that any occluding structure can be used, including for example polymers. Such polymers include woven and nonwovent polymers, natural and artificial polymers. In certain embodiments, a bioresorbable polymer is used, including, for example, extracellular matrix material (see generally, U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666, incorporated herein by reference to the extent of their technical disclosure, describing various ECM-derived matrices and methods of preparing ECM-derived matrices). In certain embodiments, the ECM is isolated from a vertebrate animal, for example and without limitation, from a warm blooded mammalian vertebrate animal including, but not limited to, human, monkey, pig, cow and sheep. The ECM can be derived from any organ or tissue, including without limitation, urinary bladder, intestine, liver, heart, esophagus, spleen, stomach and dermis. In one embodiment, the ECM is isolated from urinary bladder. Certain tissues may be superior or inferior to others in their use for the purposes described herein. Urinary bladder-derived ECM (UBM) has a smooth, relatively non-thrombogenic surface, the basement membrane; it is envisioned that this would be facing outwards towards the blood to minimize the possibility of thrombus formation. The ECM may or may not include the basement membrane portion of the ECM. In certain embodiments, the ECM includes at least a portion of the basement membrane. For instance, small intestine submucosa may not be preferred for use on a surface of a device described herein because it does not have a basement membrane and could lead to a thrombogenic response. The material used to make a device may comprise primarily (that is, greater than 50%, 60%, 70%, 80%, or 90%) ECM. This material may or may not retain some of the cellular elements that comprised the original tissue such as capillary endothelial cells or fibrocytes.

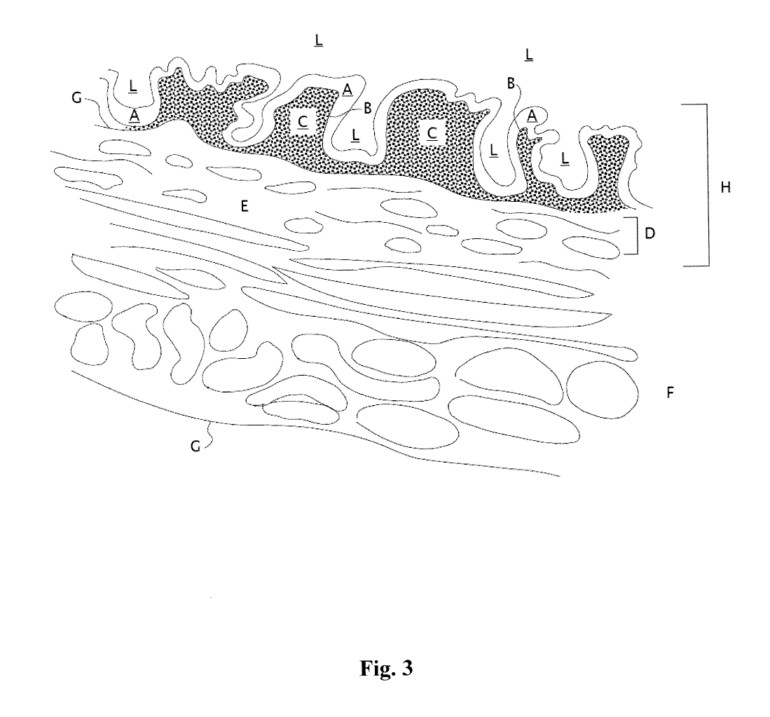

In one embodiment, the ECM is harvested from porcine urinary bladders. Briefly, the ECM is prepared by removing the urinary bladder tissue from a pig and trimming residual external connective tissues, including adipose tissue. All residual urine is removed by repeated washes with tap water. The tissue is delaminated by first soaking the tissue in a de-epithelializing solution, such as hypertonic saline, for example and without limitation, 1.0 N saline, for periods of time ranging from 10 minutes to 4 hours. Exposure to hypertonic saline solution effectively removes the epithelial cells (layer A of FIG. 3) from the underlying basement membrane (layer B of FIG. 3). The tissue remaining after the initial delamination procedure includes epithelial basement membrane and the tissue layers abluminal to the epithelial basement membrane. This tissue is next subjected to further treatment to remove the majority of abluminal tissues, but not the epithelial basement membrane. The outer serosal, adventitial, smooth muscle tissues, tunica submucosa and most of the muscularis mucosa are removed from the remaining de-epithelialized tissue by mechanical abrasion or by a combination of enzymatic treatment, hydration, and abrasion. Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. After these tissues are removed, the resulting ECM consists mainly of epithelial basement membrane and subjacent tunica propria (layers B and C of FIG. 3).

In another embodiment, the ECM is prepared by abrading porcine bladder tissue to remove the outer layers including both the tunica serosa and the tunica muscularis (layers G and F in FIG. 3) using a longitudinal wiping motion with a scalpel handle and moistened gauze. Following eversion of the tissue segment, the luminal portion of the tunica mucosa (layer H in FIG. 3) is delaminated from the underlying tissue using the same wiping motion. Care is taken to prevent perforation of the submucosa (layer E of FIG. 3). After these tissues are removed, the resulting ECM consists mainly of the tunica submucosa (layer E of FIG. 3).

The ECM can be sterilized, and typically decellularized by any of a number of standard methods without loss of its ability to induce endogenous tissue growth. For example, the material can be sterilized by propylene oxide or ethylene oxide treatment, gamma irradiation treatment (0.05 to 4 mRad), gas plasma sterilization, peracetic acid sterilization, or electron beam treatment.

The material can also be sterilized by treatment with glutaraldehyde, which causes cross linking of the protein material, but this treatment substantially alters the material such that it is slowly resorbed or not resorbed at all and incites a different type of host remodeling which more closely resembles scar tissue formation or encapsulation rather than constructive remodeling. If desired, cross-linking of the protein material can also be induced by physical and/or chemical methods, including without limitation, treatment with carbodiimide or dehydrothermal or photooxidation methods. More typically, ECM is disinfected by immersion in 0.1% (v/v) peracetic acid (a), 4% (v/v) ethanol, and 96% (v/v) sterile water for 2 h. The ECM material is then washed twice for 15 min with PBS (pH=7.4) and twice for 15 min with deionized water. Use of cross-linked ECM-derived materials for the device to produce portions of all or part of a semi-rigid or rigid frame structure may be desired. For instance, a portion of the frame of the device can be constructed from semi-rigid or rigid frame prepared from slowly-resorbable (more slowly than the ECM-derived scaffold portions of the device) cross-linked ECM-derived material(s).

In one non-limiting example of an ECM-derived material, Freytes, D. O. et al. describes preparation and testing of various types of the ECM-derived material in laminar forms (“Biaxial strength of multilaminated extracellular matrix scaffolds,” Biomaterials, 25, p. 5355-5361 (2004)). Described in that reference are methods for: harvesting ECM; preparing porcine urinary bladder submucosa ECM (UBM), porcine urinary bladder tunica propria ECM (UBS), composite porcine UBS+UBM, and canine stomach submucosa ECM (SS); disinfecting ECM-derived materials with peracetic acid treatment; preparing laminar forms of the ECM-derived materials; measuring the mechanical properties of the laminar forms; and determining cross-sectional structures of the laminar forms using scanning electron microscopy.

Commercially available ECM preparations can also be used to make a device described herein. In one embodiment, the ECM is derived from small intestinal submucosa or SIS. Commercially available preparations include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). In another embodiment, the ECM is derived from dermis. Commercially available preparations include, but are not limited to Pelvicol™ (sold as Permacol™ in Europe; Bard, Covington, Ga.), Repliform (Microvasive; Boston, Mass.) and Alloderm™ (LifeCell; Branchburg, N.J.). In another embodiment, the ECM is derived from urinary bladder. Commercially available preparations include, but are not limited to UBM (Acell Corporation; Jessup, Md.).

In further non-limiting embodiments, the ECM-derived matrix of a device described herein is seeded with cells, typically autologous or allogeneic cells, prior to or during implantation. In one example, the device is co-cultured ex vivo in a suitable bioreactor with a patient's (autologous) cells or with cells from another suitable patient (allogeneic). Suitable cells are, for example and without limitation, smooth muscle cells, bone marrow cells, cheek scrapings and biopsies from healthy cardiac, esophageal or intestinal tissue from the patient or from another patient. Cells from a patient, such as cells obtained from a biopsy of healthy tissue obtained from a patient can be seeded onto the device, for example by digesting the tissue with trypsin then resuspending the cells in media and seeding on the scaffold. Alternatively, the cells can be stem cells or other progenitor cells. Variations on these methods would be apparent to one of skill in the art.

In one embodiment, the ECM-derived material is in sheet form (see e.g. FIG. 4A). The ECM-derived material can be formed by any method. In one embodiment, the method comprises treatment with peracetic acid, lyophilization and chemical cross-linking.

In another embodiment, described in relation to FIGS. 4B-4D, the device comprises ECM-derived material in a laminar form. The laminar material comprises between 2 to 20 ECM sheets, between 4-10 ECM sheets, or 2, 3, 4, 5, 6, 7, 8, 9 or 10 ECM sheets, where each sheet typically has a thickness between 40 to 200 micrometers. Layers of sheets can be laminated together using various methods known in the art, including without limitation, treatment by vacuum-pressing, chemical bonding through cross-linking with carbodiimide or isothiocyanate or photooxidation methods, non-chemical bonding by dehydrothermal methods.

In a further embodiment, the ECM-derived material is oriented so that when the device is implanted/installed, a non-thrombogenic or less-thrombogenic (as compared to other surfaces of the ECM-derived material) surface of the ECM-derived material is exposed to the blood-stream. For UBM, the urothelial basement membrane provides a surface that minimizes both thrombogenic and immune responses by the patient.

In one embodiment shown in FIG. 4A-4D, the device comprises an ECM-derived material with a collapsible frame that is deployed at the site of the defect. As shown in FIG. 4C, the device 120 comprises a frame with distal 130 and proximal 150 frame portions to occlude a defect, a connector 140 between the distal 130 and proximal 150 frame portions that remains within the defect, and a fastener 160 to allow for retrieval of the device. As shown in FIG. 4B, multiple layers of ECM-derived material 135, 136, 137, and 138, may be used to produce distal 139 and proximal 159 sealing members (see FIGS. 4C and 4D). The ECM-derived sealing members 139 and 159 can be attached to both the distal and proximal frame portions 130 and 150. Optionally, the device may only include either the distal 139 or proximal 159 sealing member, and omit the other. The distal frame portion 130 comprises struts 132 and eyelets 131, wherein the eyelets can be used to attach a distal ECM-derived sealing member 139. The proximal portion 150 of the frame also comprises struts 152 and eyelets 151, wherein the eyelets can be used to attach a proximal ECM-derived sealing member 159.

There are several advantages of a frame having the structure as shown in FIG. 4C. The ECM can be sutured to the eyelets 151 to allow greater conformity of the frame. The ECM could be attached to the proximal portion only 150 to reduce the possibility of any particulate or thrombogenic material release. In addition to retrieval through the fastener 160, bioabsorbable sutures can be connected to all or some of the eyelets 151 on the proximal portion 150 to allow for recapture or repositioning. Twists can be incorporated into the connector 140 that can unwind to adjust for different tunnel lengths of defects and/or to allow for different tunnel widths.

The ECM-derived material can be incorporated with the collapsible frame in different ways. In one embodiment, the device comprises a collapsible frame and ECM-derived material on both the distal and proximal portions of the frame. In another embodiment, the device comprises a collapsible frame and ECM-derived material on only the proximal or distal portion of the frame.

An ECM-derived hydrogel may be incorporated into the device. In one embodiment, the device comprises a frame and an ECM-derived hydrogel injected between the distal and proximal portions. In another embodiment, the device comprises a frame that is deployed at the site of the defect and then ECM-derived hydrogel is injected into the defect with a needle in a catheter or a trocar.

As used herein, the term “ECM-derived hydrogel” and “hydrogel” refers to a gelled solubilized extracellular matrix prepared by comminuting and protease-digesting the material, and then gelling the digested material. In one non-limiting embodiment, an ECM-derived hydrogel is prepared by a method comprising: (i) comminuting an ECM-derived material, (ii) solubilizing the extracellular matrix by digestion with an acid protease in an acidic solution to produce a digest solution, (iii) raising the pH of the digest solution to a pH between 7.2 and 7.8 to produce a neutralized digest solution, and (iv) gelling the solution, typically at a temperature greater than 25° C. The ECM typically is derived from mammalian tissue, such as, without limitation from one of urinary bladder, spleen, liver, heart, pancreas, ovary, or small intestine. In certain embodiments, the ECM is derived from a pig, cow, horse, monkey, or human. In one non-limiting embodiment, the ECM is lyophilized and comminuted. The acid protease may be, without limitation, pepsin or trypsin, and in one embodiment is pepsin.

The ECM typically is solubilized at an acid pH suitable or optimal for the protease, such as greater than about pH 2, or between pH 2 and 4, for example in a 0.01M HCl solution. The solution typically is solubilized for 12-48 hours, depending upon the tissue type, with mixing (stirring, agitation, admixing, blending, rotating, tilting, etc.). Once the ECM is solubilized (typically substantially completely) the pH is raised to between 7.2 and 7.8, and according to one embodiment, to pH 7.4. Bases, such as bases containing hydroxyl ions, including NaOH, can be used to raise the pH of the solution. Likewise buffers, such as an isotonic buffer, including, without limitation, Phosphate Buffered Saline (PBS), can be used to bring the solution to a target pH, or to aid in maintaining the pH and ionic strength of the gel to target levels, such as physiological pH and ionic conditions. The neutralized digest solution can be gelled at temperatures approaching 37° C., typically at any temperature over 25° C., though gelation proceeds much more rapidly at temperatures over 30° C., and as the temperature approaches 37° C.

Any useful cytokine, chemoattractant or cells can be mixed into the composition prior to gelation or diffused, absorbed and/or adsorbed by the hydrogel after it is gelled. For example and without limitation, useful components include growth factors, interferons, interleukins, chemokines, monokines, hormones, angiogenic factors, drugs and antibiotics. Cells can be mixed into the neutralized solubilized hydrogel. When the gel is seeded with cells, the cells can be grown and/or adapted to the niche created by the ECM hydrogel by incubation in a suitable medium in a bioreactor or incubator for a suitable time period to optimally/favorably prepare the composition for implantation in a patient. For example and without limitation, the cells can be autologous or allogeneic with respect to the patient to receive the device comprising the gel. The cells can be stem cells or other progenitor cells, or differentiated cells. In one example, endothelial cells obtained from the patient are seeded on a hydrogel, for use in repairing a cardiac defect.

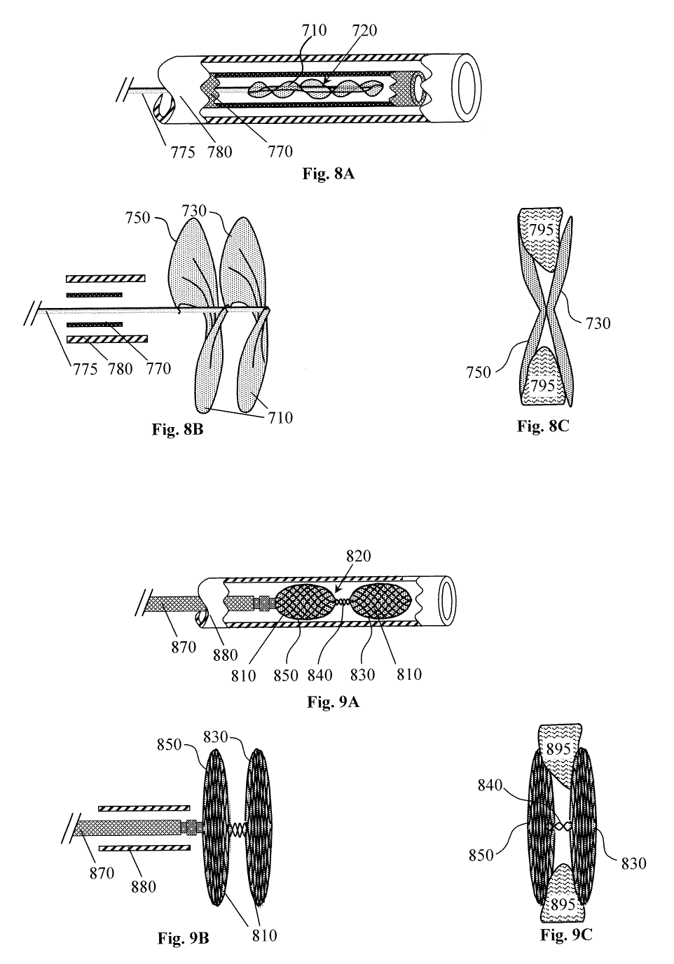

In one embodiment shown in FIGS. 8A to 8C, the device 720 comprises an ECM-derived material 710 attached to a frame with a helical periphery, similar to the GORE HELEX Septal Occluder. Currently, the GORE HELEX Septal Occluder uses an expanded PTFE material for the occlusion discs. As shown in FIG. 8A, the delivery catheter 780 contains the control catheter 770 and the collapsed device 720 comprising a frame of shape memory alloy and a central mandrel 775. To deploy the device as shown in FIG. 8B, the frame is advanced out of the delivery catheter 780 by the control catheter 770 and the central mandrel 775 is withdrawn. When the device is installed, for example and without limitation, within a defect in the septum 795 as shown in FIG. 8C, one occlusion disc 730 is within the left atrium and the other disc 750 is within the right atrium.

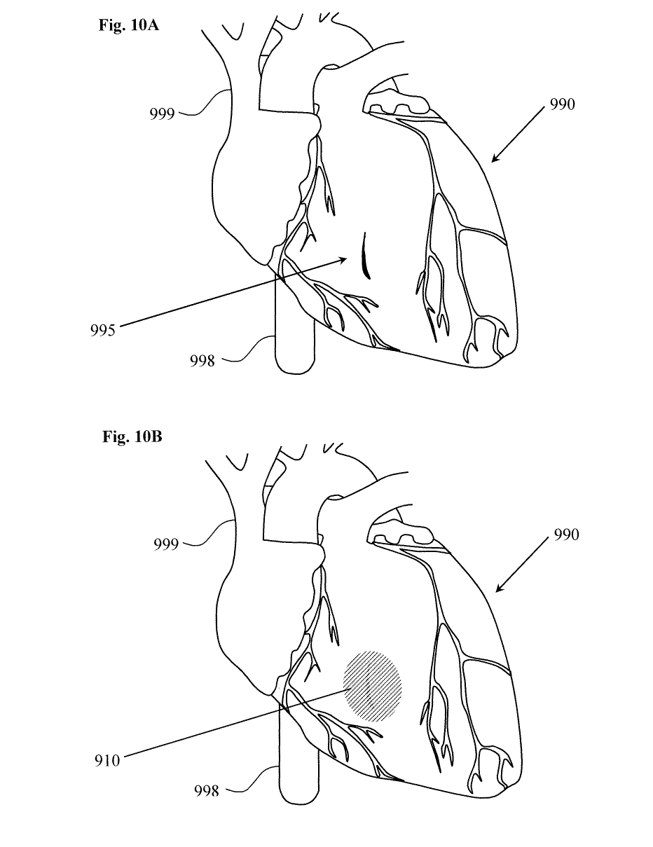

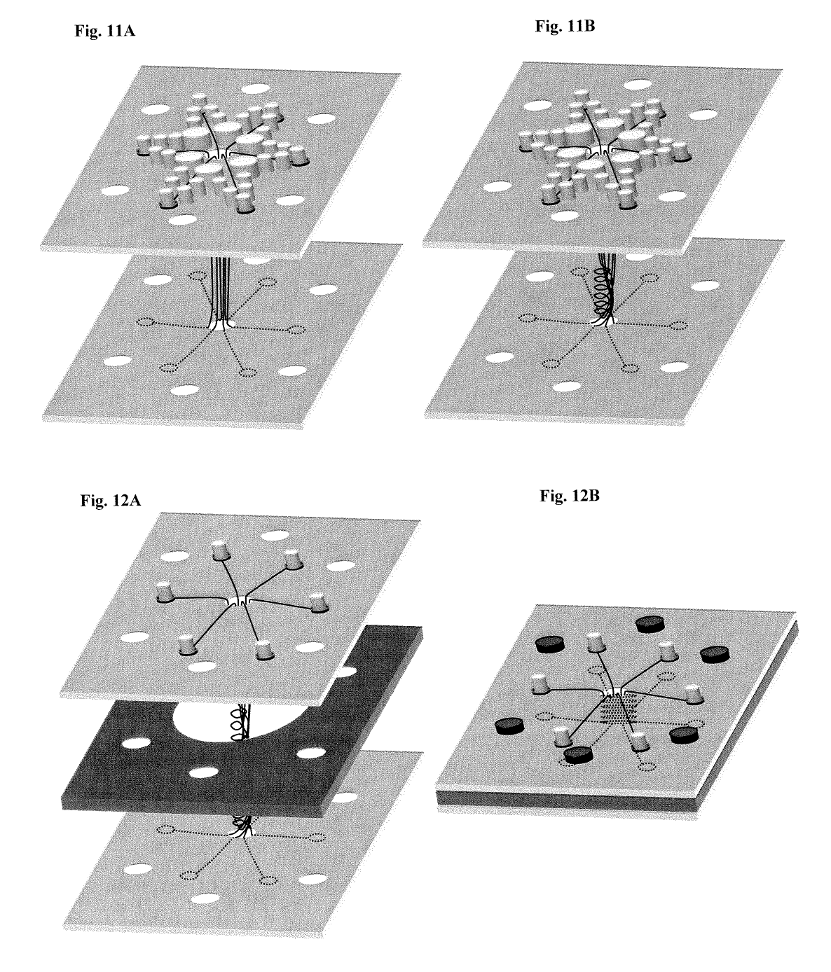

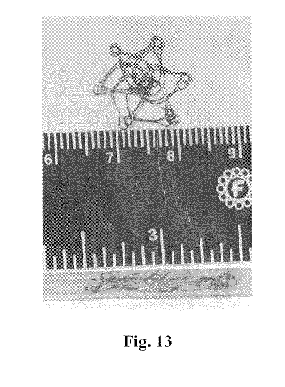

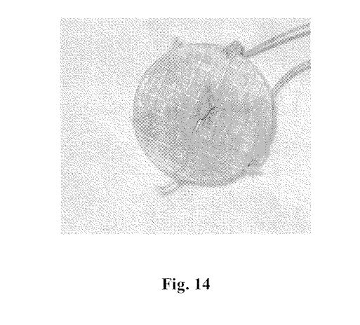

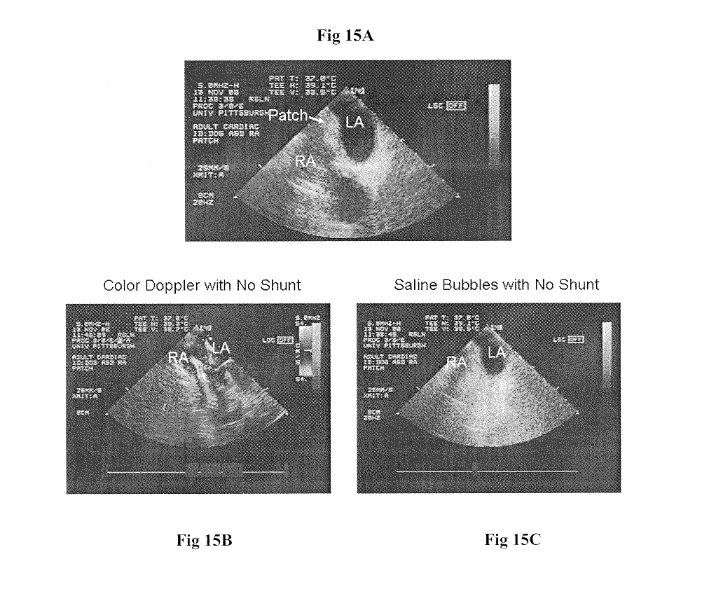

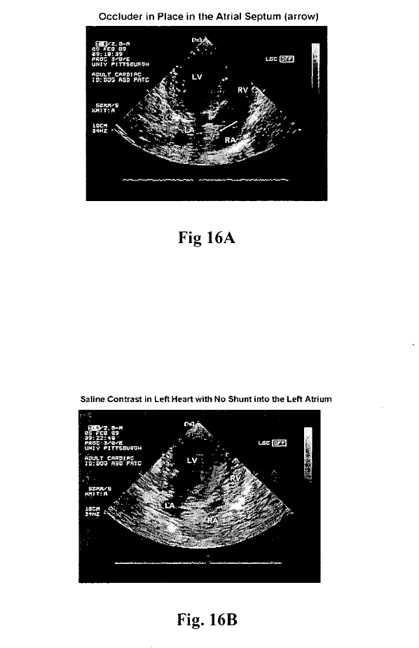

In another embodiment as shown in FIGS. 9A to 9C, the device 820 comprises an ECM-derived material 810 attached to a double disc occlusion device composed of superelastic wire mesh, like the AMPLATZER. Currently, the AMPLATZER uses a polyester fabric within the occlusion discs. As shown in FIG. 9A, a device comprises a distal occlusion disc 830, a connector 840, and a proximal occlusion disc 850. To deploy the device as shown in FIG. 9B, the distal 830 and proximal 850 discs are advanced out of the catheter 880 by the guide wire 870. When the device is installed within the defect 895 as shown in FIG. 9C, one occlusion disc 830 is within the left atrium and the other disc 850 is within the right atrium. The connector 840 between the discs spans the tunnel length of the defect in the septum 895.

Some defects within patients are not amenable for collapsible device. In one non-limiting embodiment shown in FIG. 10A, the defect is a cardiac rupture 995 within the heart 990. The inferior vena cava 998 and the superior vena cava 999 are shown for reference. In one embodiment shown in FIG. 10B, the device 910 is a patch comprising an ECM-derived material having a coating on one side comprising a medically approved adhesive. The device 910 is directly attached to the site of the defect 995. The ECM-derived patch can be attached using any number of medically accepted procedures, including but not limited to, the use of staples, sutures, or adhesives, such as fibrin or cyanoacrylate. In a further embodiment, the patch is adhered to septum on right atrium wall and the non-thrombogenic or less thrombogenic surface of the patch faces away from the septum.

The device can also be available in a kit for cardiac repair. In a broad embodiment, the kit comprises a device in any embodiment described herein, comprising hydrated or dehydrated ECM-derived material in any commercially and medically acceptable container. An acceptable container includes, without limitation, a box, a package, a bubble-pack, a foil and/or plastic pouch, can be vacuum-sealed, and is preferably packaged in a sterile condition. The device can be packaged in the expanded condition or collapsed configuration. The device can be treated in any methods known in the art, such as without limitation, dehydration by lyophilization or exposure to low-humidity vacuum; sterilization by treatment with propylene oxide or ethylene oxide, gamma irradiation treatment (0.05 to 4 mRad), gas plasma sterilization, peracetic acid sterilization, or electron beam treatment.

The kit for cardiac repair can also include catheter(s), trocar(s), cannula(e) or guide wires to aid in delivery of the device. In one embodiment, the kit comprises a device comprising dehydrated ECM-derived material and frame and a guide wire, wherein the device is attached to the guide wire. The fastener on the device and the locking mechanism on the guide wire are complementary portions of a clasp system. For example and without limitation, the fastener can be a threaded bore or nut and the locking mechanism can be configured to be a bolt that engages the threaded bore. In another embodiment, the kit further comprises a device comprising dehydrated ECM-derived material, a guide wire and a guiding catheter, wherein the device is attached to the guide wire and contained within the lumen of the guiding catheter.

To use the kit, the operator would insert a delivery catheter within the patient to access the defect or hole. The delivery catheter typically would be less than 10 French to aide navigation. The operator would then hydrate device from the kit, if it is dehydrated, and guide the device into the delivery catheter, a funnel could be used. The device can be hydrated in an isotonic, buffered PBS solution or any solution known in the art immediately prior to implantation. A guide wire and guiding catheter could be used to aide navigation of the device through the delivery catheter and to deploy the device at the site of the defect.

In one embodiment, the kit is used to repair a defect that is an atrial septal defect. The delivery catheter may be inserted into the femoral vein, up the vena cava, into the right atrium, and through the ASD into the left atrium. The device would be hydrated, if needed, and then pushed through the delivery catheter by a guide wire. The distal portion of the device is pushed out of the delivery catheter and deployed in the left atrium. The delivery catheter would then be pulled back to deploy the proximal portion of the device in the right atrium. The device could be withdrawn if placement is not optimum by using the suture attached to the eyelets of the frame or by using the fastener on proximal frame portion.

When the kit comprises a device, a guide wire or guiding catheter can be attached to the fastener of the device and then pushed through the lumen of the delivery catheter. When the kit comprises a device connected to a guide wire, the device with the guide wire is inserted into the delivery catheter and delivered to the site of the defect. When the kit comprises a device connected to a guide wire within a guiding catheter, the guide catheter can be inserted into the delivery catheter and guided to the site of the defect. At the site of the defect, the device is deployed and released from the guide wire.

In a further non-limiting embodiment, the kit for cardiac repair can also include ECM-derived hydrogel in any commercially and medically acceptable container, such as, without limitation, a gel pack. In use, the operator can, without limitation, inject the ECM-derived hydrogel between the distal and proximal portions of the device before implantation. In another non-limiting embodiment, the operator can inject the ECM-derived hydrogel into the defect with a needle in a catheter or a trocar after implantation of the device. The hydrogel can be partially or fully gelled before injection to reduce or prevent leakage from the device into the bloodstream.

The following Examples are provided for illustration and, while providing specific example of embodiments described herein, are not intended to be limiting.

### EXAMPLES

### Example 1

### Preparation of Porcine Extracellular Matrix-Derived Urinary Bladder Matrix (UBM)

To prepare porcine UBM, urinary bladders were harvested, cleaned and rinsed. Adipose and connective tissues were trimmed from the edges and the outer surface of the bladder. The apex of the bladder was cut off about half an inch above the tail and the bladder was sliced length-wise. The abluminal tissues were loosened and removed. Cuts were made into the muscularis externa and submucosal layers of the bladder tissue. Muscle layers, including the muscularis mucosa, were pulled away by forceps. The final product contained mainly tunica propria and the underlying basement membrane. After inspection, additional muscle tissues were removed and the UBM was stored in type 1 water in 4° C.

To disinfect and depyrogenate the UBM, excess fluid was removed from the stored UBM by squeezing or mechanical wringing and by placing on an absorbent surface. The composition of the peracetic acid solution should be approximately 0.1% (v/v) peracetic acid in 4% (v/v) ethanol and 96% (v/v) sterile water. The UBM and peracetic acid solution was placed on a shaker for two hours. The UBM was then washed twice for 15 min with PBS (pH=7.4) and twice for 15 min with deionized water. Finally, the UBM was lyophilized to dry the sheet.

### Example 2

### Preparation of the Wire Frame and Assembly of the Device with UBM

The frame portions of the device were prepared by using a series of forming tools to preset the shape of the struts, eyelets and connectors. The first set of forming tool comprises pegs to establish the shape of the proximal and distal frame portions and of the connector. The second set of forming tools comprises jigs to compress the connector portion of the device while maintaining the shapes of the distal and proximal frame portions.

As shown in FIGS. 11A and 11B, the first set of forming tools comprised two plates, where the top plate and bottom plate were identical. As in FIG. 11A, each plate had pegs patterned in the desired shape of the device and six holes on the periphery of the pegs to accommodate threaded rods. The center hole of the plate allowed for wire to be passed between the two plates. 0.010 inch NiTi SE 508 wire was wound around the pegs to form the frame of the device. Threaded rods were inserted into the holes to hold the two plates together, where the distance between the plates was approximately 10 millimeters. The forming tools and the wire frame were placed in a furnace at 550° C. for 10 minutes, followed by a water quench. The threaded rods were removed from the plates.

As shown in FIG. 11B, twists were introduced into the connector by rotating the top plate by approximately 540° or 1.5 complete turns. The threaded rods were replaced within the holes in the plate to maintain the twisted shape of the connector during heat treatment. The forming tools and the wire frame were again placed in a furnace at 550° C. for 10 minutes, followed by a water quench.

As shown in FIGS. 12A and 12B, the second set of forming tools comprised three plates to compress the connector portion of the device while maintaining the shapes of the distal and proximal frame portions. As in FIG. 12A, the top and bottom plates were identical and had a peg for each eyelet, while the middle plate had a large center hole. The eyelets of the distal portion of the frame were guided onto the pegs of the top plate. The connector was inserted into the large center hole within the middle plate and the eyelets of the proximal portion of the frame were guided onto the pegs of the bottom plate. As shown in FIG. 12B, threaded rods were inserted into all three plates to compress the connector portion of the frame. The plates and the wire frame were placed in a furnace at 550° C. for 10 minutes, followed by a water quench. The wire frame was removed from the forming tools. The Austenite Final temperature was verified to be below 37° C., where the wire frame was chilled to 0° C. and deformed and the frame fully recovered its shape when warmed to 37° C.

Porcine ECM-derived Urinary Bladder Matrix (UBM) was prepared as explained in Example 1. A four-layer lyophilized sheet was sutured to the eyelets on the proximal and distal portion of the frame with three half loops of 5-0 Ti-cron sutures. The less-thrombogenic basement membrane of the UBM was pointing away from the frame. The three half loops were tied together in the center along with a platinum radiopaque marker. A single loop of suture was made through all the eyelets on the distal frame portion so that the device could be drawn into a catheter. The device was then packaged into a catheter and sterilized using electron beam treatment, gamma irradiation or ethylene oxide treatment.

### Example 3

### In Vivo Testing

The ability of an extracellular matrix scaffold to function as a repair device for experimentally produced atrial septal defects (ASD) was studied in a dog model. The device was manufactured from a four layers of vacuum pressed urinary bladder matrix (UBM). This study evaluated the ability for the UBM device to prevent blood flow shunting as a result of the created ASD as well as the morphology of the atrial free wall at 3 months. In addition, histology of the patched areas was evaluated at the 3 month timepoint. A prototype for the percutaneous delivery of the UBM ASD patch was also created. The prototype was optimized based on in vivo work and benchtop testing. Specifically, a NiTi frame with self sizing waist region was developed. The wire was made in a multi step forming process so that the middle waist region would compress to the thickness and width of the septal wall defect. The ECM was attached in a way such that no force was transmitted to the ECM loading into or deployment from the delivery system.

Each animal was fed appropriate amounts of dog food. The dogs were supplied with tap water ad libitum. A four layer UBM patch with luminal layer facing outward on both surfaces was prepared as in Example 1.

Dogs were anesthetized (sodium thiopental, 12-25 mg/kg IV for induction and intubation. Animals were then be maintained at a surgical plane of anesthesia with Isoflurane (1-3% in oxygen). Blood pressure (via femoral artery) and ECG was monitored throughout the surgical procedure. The animals were infused with 2 ml/kg/h of lactated Ringer's solution or equivalent solution throughout the procedure.

Prior to undergoing thoracotomy the wound edges were infiltrated with local anesthetic (marcaine or bupivicaine, ˜10-15 ml) effectively blocking the intercostal nn. A right thoracotomy was made at the third intercostal space, followed by a pericardiotomy and placement of suspension sutures to cradle the heart. Visualization of the heart, the pulmonary valve outflow tract, aorta, and right atrium was accomplished. Heparin was administered IV (25-75 IU/kg). The animal was then placed on cardiopulmonary bypass (CPB) by cannulation of the vena cava and the outflow cannulae for the cardiopulmonary bypass was inserted into either the carotid artery using a cutdown or into the aorta based on the individual anatomy of the animal. Ventricular fibrillation was induced by standard cardioplegia.

For the creation of an ASD the right atrium was opened and a portion of the intra-atrial septum in the fossa ovalis was excised (approximately 2 cm×2 cm). The defect was repaired using a ECM scaffold material. The ECM scaffold device was sewn into its place with 7-0 non-absorbable suture material (e.g. Prolene). The hole in the right atrium was closed with ECM scaffold in the same manner as the ASD. At the conclusion of surgery, defibrillation was achieved and the dogs were weaned from CPB. A chest tube was placed prior to closing the chest and maintained up to 72 hours to ensure negative pressure compliance in the chest and to remove any excess drainage present after a procedure of this type. The chest wall was closed using routine thoracic closure technique (1-0 Prolene for closure of the ribs, 2-0 PDS for SQ and 2-0 Prolene or staples for skin closure). Skin staples/sutures were removed 10 days post-op.

Following the surgical procedure and cessation of inhalation anesthesia, animals were continually monitored for 24 hours, recording the following parameters every hour: pulse rate, strength of pulse, capillary refill time, amount of fluid removed from chest via the chest drain, respiratory rate & ability to maintain an open airway, urinary output, and defecation. Body temperature was determined and recorded every 2 hours.

Extubation was based on the presence of a swallowing reflex and protective cough reflexes that are functional. The pulse, respiration, body temperature, jaw tone, capillary refill time, and mucous membrane color was evaluated prior to removing the endotracheal tube. Dogs were held in a recovery cage for up to 72 hours. The dogs were moved to a run when they demonstrated normal respiration, did not demonstrate pain, being bright, alert, and responsive. At this time the cephalic vein line was removed.

Non invasive echocardiograms were performed at 1 week and at the time of sacrifice. In addition, the implants were harvested after euthanasia for mechanical properties testing and macroscopic and microscopic examination. The measured endpoints were evaluated at the following time point: 3 months. Buprenorphine hydrochloride (dogs, 0.01-0.02 mg/kg, SQ, q12 h; pigs, 0.005-0.01 mg/kg, IM or IV, q12 h), was administered at regular intervals for 4 days for pain, then was continued to be administered for pain management if signs of pain are exhibited. Aspirin (325 mg/day) was given for the duration of the study, administered as anticoagulant therapy.

Following the first 24 hours, the animals were evaluated and assessed for the need for additional continuous monitoring. If an animal was unstable (unable to maintain a stable pulse, respiration, clotting time, hematocrit), continual monitoring would follow for an additional 24 hours. Once an animal would be considered stable, monitoring frequency would decrease to once every 2-4 hours, then once every 4-12 hours, and finally, once every 24 hours.

At three months following surgery, a final echocardiogram was performed prior to euthanasia. Euthanized graft sites were analyzed grossly and tissues harvested for morphologic evaluation. Specifically, at the 3 month time point animals were evaluated for flow from the left and right atrium as well as visually inspected. Heparin was administered IV (110-500 IU/kg). A sternotomy, followed by a pericardiotomy and placement of suspension sutures to cradle the heart. Visualization of the heart, the pulmonary valve outflow tract, aorta, and right atrium was accomplished. Trans esophageal echocardiogram was used to visualize the defect. Isoflurane was increased to 5% for 5 minutes. The vena cavas, pulmonary arteries, and aorta were clamped. The heart was then excised and perfusate flushed through. The scaffold placement site and the adjacent native tissue was excised, divided, and placed in neutral buffered formalin for routine H&E and Masson's Trichrome staining or 4% Paraformaldehyde for immunofluorescence.

The first two attempts to create the defect in a dog were unsuccessful. During the first surgery the AV node was crushed creating the ASD defect and during the second surgery a vein was irreparably punctured during the cannulation. On the third attempt, a 10 mm patch was placed in the septal wall and a 30 mm patch on the atrial free wall. The rehydrated device was easy to manipulate and suture. Both patches were competent at initial surgery and at 3 months. There was no shunting between the atriums as determined by microbubble test at 1 week or 3 months. The UBM ECM patches had smooth intact endothelialized non-thrombogenic surface. The patched areas were well vascularized and integrated into the adjacent myocardium. The device was replaced by a mixture of connective tissues: dense collagenous tissue and adipose tissue. The freewall also had small islands of muscle and fingers of muscle from the adjacent native tissue. There were also some chondrocytes in the freewall. The results show that the occluding device was clinically successful.

Having described this invention above, it will be understood to those of ordinary skill in the art that the same can be performed within a wide and equivalent range of conditions, formulations and other parameters without affecting the scope of the invention or any embodiment thereof. Any document incorporated herein by reference is only done so to the extent of its technical disclosure and to the extent it is consistent with the present application and the disclosure provided herein.

1. An occluding device, comprising an occluding member attached to a collapsible frame and the collapsible frame further comprising a distal sealing portion, a proximal sealing portion, a connector between the sealing portions and a fastener attached to the proximal sealing portion and/or the connector, wherein the fastener allows for manipulation and retrieval of the device.

2. The device of claim 1, wherein the fastener is a threaded bore or a bolt from a bolt-and-nut type clasp.

3. The device of claim 1, wherein the collapsible frame is an elastic material.

4. The device of claim 3, wherein the elastic material comprises a metal.

5. The device of claim 4, wherein the metal is an elastic wire.

6. The device of claim 5, wherein the elastic wire is a nickel-titanium alloy.

7. The device of claim 1, wherein the distal and proximal sealing portion are a polymer.

8. The device of claim 1, wherein the distal and proximal sealing portion are ECM-derived material.

9. The device of claim 8 wherein the elastic material is cross-linked extracellular matrix tissue.

10. The device of claim 1, wherein the proximal and distal sealing portions are cambered.

11. The device of claim 1, wherein the proximal and distal sealing portions comprise eyelets.

12. The device of claim 11, wherein a grafting material is attached to the eyelets on the proximal portion of the frame.

13. The device of claim 12, wherein the eyelets are configured to allow for manipulation and retrieval of the device.

14. The device of claim 13, wherein the eyelets comprise sutures.

15. The device of claim 1, wherein the connector between the proximal and distal portions of the frame comprises one or more shape memory fibers.

16. The device of claim 1, wherein the connector consists of a single spring-shaped memory fiber.

17. The device of claim 16, were the connector comprises a plurality of shape memory fibers extending from the proximal to the distal portion and formed into a preset shape of a twisted bundle, and which can be untwisted by rotating one or both of the proximal and distal portions relative to each other.

18. The device of claim 17, wherein the shape memory fibers comprise a material selected from the group consisting of nitinol, polylactide, and magnesium alloy.

19. The device of claim 18, wherein magnesium alloy is an absorbable metal.

20. The device of claim 1, wherein the device comprises a grafting material.

21. The device of claim 20, wherein the grafting material comprises extracellular matrix (ECM) tissue isolated from urinary bladder tissue.

22. The device of claim 21, wherein the extracellular matrix (ECM) tissue isolated from urinary bladder tissue and having an abluminal side is fixed such that the abluminal side is facing away from the device.

23. A method of treating a patient having a heart defect, comprising implanting the device of claim 1 in the heart of the patient.

24. The method of claim 23, wherein the heart defect of the patient is an atrial septal defect (ASD).
